(19) United States
(12) Patent Application Publication
(10) Pub. No.: US 2006/0253085 A1
(43) Pub. Date: Nov. 9, 2006

(54) Dual insertion set

(75) Inventors: Eric P. Geismar, Encino, CA (US); Bradley J. Enegren, Moorpark, CA (US); Paul H. Kovelman, Simi Valley, CA (US)

Correspondence Address: MEDTRONIC MINIMED INC., 18000 DEVONSHIRE STREET, NORTHRIDGE, CA, 91325-1219, US

(73) Assignee: MEDTRONIC MINIMED, INC.

(21) Appl. No.: 11/149,119

(22) Filed: Jun. 8, 2005

Related U.S. Application Data
(60) Provisional application No. 60/678,861, filed on May 6, 2005.

(51) Int. Cl. A61M 5/32; A61B 5/00

(52) U.S. Cl. 604/272; 600/327

(57) ABSTRACT

A dual insertion set includes a base, an infusion portion, a sensor portion, and at least one piercing member. The base is adapted to secure the dual insertion set to the skin of a patient. The infusion portion includes a cannula for supplying a fluid to a placement site. The cannula is coupled to and extends from the base and has at least one lumen with a distal end for fluid communication with the placement site. The cannula has at least one port structure formed near another end of the lumen opposite the distal end. The sensor portion includes a sensor coupled to and extending from the base having at least one sensor electrode formed on a substrate. The sensor is for determining a body characteristic of the patient at the placement site. The at least one piercing member is coupled to and extends from the base to facilitate insertion of the cannula and the sensor.

### RELATED APPLICATIONS

This application claims the benefit of prior filed U.S. Provisional Application Ser. No. 60/678,861, filed May 6, 2005.

### FIELD OF THE INVENTION

Embodiments of the invention relate to improved sensor and infusion devices with improved placement aspects and, more particularly, to devices and methods for placing a sensor at a selected insertion site within the body of a patient, in addition to placing an infusion set with a catheter for delivery of selected fluids.

### BACKGROUND OF THE INVENTION

Insulin must be provided to people with Type 1 and many with Type 2 diabetes. Traditionally, since it cannot be taken orally, insulin has been injected with a syringe. More recently, use of external infusion pump therapy has been increasing, especially for delivering insulin for diabetics using devices worn on a belt, in a pocket, or the like, with the insulin delivered via a catheter with a percutaneous needle or cannula placed in the subcutaneous tissue. For example, as of 1995, less than 5% of Type 1 diabetics in the United States were using pump therapy. There are now about 12% of the currently over 1,000,000 Type 1 diabetics in the U.S. using insulin pump therapy, and the percentage is now growing at an absolute rate of over 2% each year. Moreover, the number of Type 1 diabetics is growing at 3% or more per year. In addition, growing numbers of insulin using Type 2 diabetics are also using external insulin infusion pumps. Physicians have recognized that continuous infusion provides greater control of a diabetic's condition, and are also increasingly prescribing it for patients. In addition, medication pump therapy is becoming more important for the treatment and control of other medical conditions, such as pulmonary hypertension, HIV and cancer.

Pump therapy systems have been developed that deliver medication by infusion into subcutaneous tissue using an infusion set with needles and/or a soft cannula. The soft cannula of the infusion set is normally inserted into the skin with a needle to prevent kinking of the soft cannula. Automatic insertion devices have been utilized to reduce the discomfort and pain involved with the insertion of infusion sets.

In addition to delivering medication to a patient, other medical devices have been used to determine body characteristics by obtaining a sample of bodily fluid. A variety of implantable electrochemical sensors have been developed for detecting and/or quantifying specific agents or compositions in a patient's blood. For instance, glucose sensors have been developed for use in obtaining an indication of blood glucose levels in a diabetic patient. Such readings can be especially useful in monitoring and/or adjusting a treatment regimen that typically includes the regular administration of insulin to the patient. Thus, blood glucose readings are particularly useful in improving medical therapies with semi-automated medication infusion pumps of the external type, as generally described in U.S. Pat. Nos. 4,562,751; 4,678,408; and 4,685,903; or automated implantable medication infusion pumps, as generally described in U.S. Pat. No. 4,573,994, all of which are specifically incorporated by reference herein.

### SUMMARY OF THE DISCLOSURE

According to an embodiment of the invention, a dual insertion set is for supplying a fluid to the body of a patient and for monitoring a body characteristic of the patient. The dual insertion set includes a base, an infusion portion, and a sensor portion. The base may be used to secure the dual insertion set to the skin of a patient. The infusion portion has at least one cannula for supplying the fluid to an infusion placement site, which is coupled to and extends from the base. The at least one cannula has at least one lumen with a distal end for fluid communication with the placement site and at least one port structure formed near another end of the at least one lumen opposite the distal end. The sensor portion has at least one sensor coupled to and extending from the base having at least one sensor electrode formed on a substrate. The at least one sensor is for determining at least one body characteristic of the patient at a sensor placement site.

Other embodiments include at least one piercing member. The at least one piercing member is coupled to and extends from the base to facilitate insertion of the at least one cannula and/or the at least one sensor.

Alternative embodiments have the infusion portion and the sensor portion spaced a predetermined distance apart from one another. Additional embodiments include at least two separate piercing members to insert the at least one cannula and the at least one sensor into the body of the patient. Further embodiments have the infusion portion and the sensor portion equal in length. Other embodiments have the length of the sensor portion sized smaller relative to the length of the infusion portion. Still additional embodiments have the length of the infusion portion sized smaller relative to the length of the sensor portion. Particular embodiments provide metal needles as the piercing members.

Further embodiments provide a cannula that includes an outer lumen to supply the fluid and an inner lumen to contain the sensor portion. The outer lumen may be sealed off at the distal end and the inner lumen may be open to allow the at least one sensor to protrude out of the inner lumen. In alternate embodiments, the outer lumen may contain the sensor portion and the inner lumen may supply the fluid. In still other embodiments, the cannula may include side-by-side lumens. The at least one cannula may also include at least one opening for infusing the fluid into the body of the patient. Additionally, one piercing member may be used to insert the dual insertion set into the body of the patient. Other embodiments may provide a sensor that includes at least one internal power supply. The internal power supply may further drive a leak detection system. Particular embodiments provide insulin as the infused fluid. In other embodiments, the monitored body characteristic may be blood glucose.

According to another embodiment of the invention, a dual insertion set is for supplying a fluid to a body of a patient and for monitoring a body characteristic of the patient including a base, an infusion portion, a sensor portion and a piercing member. The base is used to secure the dual insertion set to the skin of a patient. The infusion portion includes at least one cannula for supplying a fluid to an infusion placement site, which is coupled to and extends from the base. The at least one cannula has at least one lumen with a distal end for fluid communication with the placement site and at least one port structure formed near another end of the at least one lumen opposite the distal end. The sensor portion includes at least one sensor having at least one sensor electrode formed on a substrate. The at least one sensor is for determining at least one body characteristic of the patient at a sensor placement site. The piercing member is coupled to and extends from the base to facilitate insertion of the at least one cannula and the at least one sensor. The at least one cannula of the illustrated embodiment may also include an outer lumen to supply the fluid and an inner lumen to contain the sensor portion. The outer lumen is sealed at the distal end and the inner lumen is open to allow the at least one sensor to protrude out of the inner lumen. The at least one cannula may also include at least one opening for infusing the fluid into the body of the patient. In some embodiments, the piercing member is a metal needle and the infused fluid is insulin. In other embodiments, the at least one monitored body characteristic is blood glucose. Additional embodiments may include an internal power supply for the at least one sensor. In further embodiments, the internal power supply may drive a leak detection system.

According to yet another embodiment of the invention, a dual insertion set is for supplying a fluid to a body of a patient and for monitoring a body characteristic of the patient including a base, an infusion portion, a sensor portion and a piercing member. The base is used to secure the dual insertion set to the skin of a patient. The infusion portion includes at least one cannula for supplying a fluid to an infusion placement site, which is coupled to and extends from the base. The at least one cannula has at least one lumen with a distal end for fluid communication with the infusion placement site and at least one port structure formed near another end of the at least one lumen opposite the distal end. The sensor portion includes at least one sensor having at least one sensor electrode formed on a substrate. The at least one sensor is for determining at least one body characteristic of the patient at a sensor placement site. The piercing member is coupled to and extends from the base to facilitate insertion of the at least one cannula and the at least one sensor. The at least one sensor may be coupled to an outer wall of the at least one cannula.

According to an alternative embodiment of the invention, a dual insertion set is for supplying a fluid to a body of a patient and for monitoring a body characteristic of the patient including a base, an infusion portion, a sensor portion and a piercing member. The base is used to secure the dual insertion set to the skin of a patient. The infusion portion includes a piercing member for penetrating the skin of the patient and for supplying a fluid to a placement site. The piercing member is coupled to and extends from the base. Additionally, the piercing member has at least one lumen with a distal end for fluid communication with an infusion placement site and at least one port structure formed near another end of the at least one lumen opposite the distal end. The sensor portion includes at least one sensor coupled to and extending from the base having at least one sensor electrode formed on a substrate. The at least one sensor is for determining at least one body characteristic of the patient at a sensor placement site. The at least one sensor is coupled to an outer wall of the piercing member.

According to a further embodiment of the invention, a dual insertion set is for supplying a fluid to a body of a patient and for monitoring a body characteristic of the patient including a base, an infusion portion, a sensor portion and a piercing member. The base is used to secure the dual insertion set to the skin of a patient. The infusion portion includes a piercing member for penetrating the skin of the patient and for supplying a fluid to an infusion placement site. The piercing member is coupled to and extends from the base. Additionally, the piercing member has at least one lumen with a distal end for fluid communication with the infusion placement site and at least one port structure formed near another end of the at least one lumen opposite the distal end. The sensor portion includes at least one sensor having at least one sensor electrode formed on a substrate. The at least one sensor is placed on the skin of the patient and is for determining at least one body characteristic of the patient at a sensor placement site. In some embodiments, a piercing member may be used to withdraw fluid from the body of the patient to provide fluid contact with the at least one sensor electrode.

Other features and advantages of the invention will become apparent from the following detailed description, taken in conjunction with the accompanying drawings which illustrate, by way of example, various features of embodiments of the invention.

### BRIEF DESCRIPTION OF THE DRAWINGS

A detailed description of embodiments of the invention will be made with reference to the accompanying drawings, where like numerals designate corresponding parts or cross-sections in the several figures.

FIG. 1 is a side view of a dual insertion set in accordance with an embodiment of the invention.

FIG. 2 is a side view of a dual insertion set in accordance with another embodiment of the invention where a sensor and a cannula are placed at different depths in the body of a patient.

FIG. 3 is a side view of a dual insertion set in accordance with a further embodiment of the invention where a sensor and a cannula are laterally spaced close to each other.

FIGS. 4(a) and 4(b) are side and front views of a dual insertion set in accordance with an alternative embodiment of the invention where a cannula includes an outer lumen and an inner lumen, where a sensor is contained within the inner lumen of the cannula, which is sealed at its distal end.

FIGS. 5(a) and 5(b) are side and front views of a dual insertion set in accordance with a further embodiment of the invention where a sensor portion is contained within an infusion portion, which is not sealed at its distal end.

FIG. 6 is a side view of a dual insertion set in accordance with another embodiment of the invention where a sensor is coupled to the infusion portion.

FIG. 7 is a side view of a dual insertion set in accordance with another embodiment of the invention where a piercing member replaces a cannula.

FIG. 8 is a side view of a dual insertion set in accordance with another embodiment of the invention where a sensor is placed above the skin of a patient.

FIG. 9 is a simplified block diagram of a dual insertion set coupled to an infusion pump in accordance with an embodiment of the invention.

### DETAILED DESCRIPTION OF THE PREFERRED EMBODIMENTS

As shown in the drawings for purposes of illustration, the invention is embodied in a dual insertion set for supplying fluids to the body of patient in addition to monitoring body characteristics. In particular embodiments, the invention is embodied in a system for regulating the rate of insulin infusion into the body of a patient based on a glucose concentration measurement taken from the body. Embodiments of the invention may be employed in various infusion environments including, but not limited to a biological implant environment. Other environments include, but are not limited to external infusion devices, pumps, or the like.

In some embodiments, the dual insertion set infuses a fluid, such as medication, chemicals, enzymes, antigens, hormones, vitamins or the like, into a body of a patient. In particular embodiments of the invention, the dual insertion set may be coupled to an external infusion device, which includes an RF programming capability, a carbohydrate (or bolus) estimation capability and/or vibration alarm capability, as described in U.S. Pat. No. 6,554,798 entitled “External Infusion Device with Remote Programming, Bolus Estimator and/or Vibration Alarm Capabilities,” which is specifically incorporated by reference herein. In other embodiments, the dual insertion set may be coupled to other infusion pumps such as the Animas IR-1250, the Deltec Cozmo®, the Disetronic D-Tron™plus, the MiniMed Paradigm® 515/715, and the Dana Diabecare® II. When coupled to an external infusion device, the dual insertion set may also include a disconnect cable, allowing the patient to easily disconnect the dual insertion set from the external infusion device to go swimming, take a shower or the like, without having to entirely remove the dual insertion set from the body of the patient. Particular embodiments are directed towards use in humans; however, in alternative embodiments, the dual insertion set may be used in animals.

In further embodiments, the dual insertion set may be adapted to fit in an insertion tool, as described in U.S. Pat. No. 5,851,197 entitled “Injector For A Subcutaneous Infusion Set,” U.S. Pat. No. 6,093,172 entitled “Injector For A Subcutaneous Insertion Set,” and U.S. Pat. No. 6,607,509 entitled “Insertion Device For An Insertion Set And Method Of Using The Same,” all of which are specifically incorporated by reference herein. The dual insertion may be further adapted for low profile and unobtrusive placement on the patient. In other embodiments, the shape of the dual insertion set may be rectangular, circular, square or the like.

A sensor included in the dual insertion set may be implanted in and/or through subcutaneous, dermal, sub-dermal, inter-peritoneal or peritoneal tissue. In other embodiments of the invention, the sensor may be coupled to a monitor for determining glucose levels in the blood and/or body fluids of the patient without the use of, or necessity of, a wire or cable connection between the transmitter and the monitor. In these embodiments, the sensor utilizes glucose oxidase to determine glucose levels. In still further embodiments, the sensor may use other materials such as optical, fluorescence or electrical materials to determine glucose levels. It will be recognized that further embodiments of the invention may be used to determine the levels of other agents, characteristics or compositions, such as hormones, cholesterol, medication concentrations, pH, oxygen saturation, viral loads (e.g., HIV), or the like. In other embodiments, the sensor may also include the capability to be programmed or calibrated using data received by a telemetered characteristic monitor transmitter device, or may be calibrated at the monitor device (or receiver), as described in U.S. Pat. No. 6,809,653 entitled “Telemetered Characteristic Monitor System And Method Of Using The Same,” which is specifically incorporated by reference herein. The telemetered characteristic monitor system may be primarily adapted for use in subcutaneous human tissue. However, still further embodiments may be placed in other types of tissue, such as muscle, lymph, organ tissue, veins, arteries or the like, and used in animal tissue. Embodiments may provide sensor readings on an intermittent, near-continuous and/or continuous basis.

In some embodiments of the invention, the dual insertion set may be coated with medications or other agents that inhibit infection and/or promote healing of the insertion site, as described in U.S. Pat. No. 5,505,713 entitled “Indwelling Catheter With A Stable Enzyme Coating,” U.S. Pat. No. 6,475,196 entitled “Subcutaneous Infusion Cannula,” U.S. Pat. No. 6,770,729 entitled “Polymer Compositions containing Bioactive Agents and Methods for Their Use,” and U.S. Patent Application Publication No. 20030199837 entitled “Anti-Inflammatory Biosensor For Reduced Biofouling And Enhanced Sensor Performance,” all of which are specifically incorporated by reference herein. Particular embodiments of the dual insertion set are for transcutaneous placement of the dual insertion set in subcutaneous tissue. In still further embodiments, the sensor portion and infusion portion of the dual insertion may be placed at different depths within the body of the patient.

The dual insertion set may be used to monitor body characteristics of the patient. In one embodiment, the sensor portion of the dual insertion set monitors blood glucose levels and can be used in conjunction with automated and/or semi-automated medication infusion pumps. In additional embodiments, the sensor portion may be used to determine the levels of other agents, characteristics or compositions, such as hormones, cholesterol, medication concentrations, pH, oxygen saturation, viral loads (e.g., HIV), or the like.

The infusion portion of the dual insertion set may be used to provide fluids to the body of a patient. In one embodiment, the infusion portion provides insulin to a diabetic patient. In other embodiments, the infusion portion provides medication, chemicals, enzymes, antigens, hormones, vitamins or the like, to the body of the patient.

In particular embodiments, the dual insertion set includes at least one piercing member to pierce the skin during insertion. The piercing member may be a metal needle, hollow, solid, half needle (or other fraction) or the like having a diameter in the range of 18 gauge—29 gauge, or the like, or any range there between. In other embodiments, the piercing member may be made out of other materials, such as ceramic, plastic, composites, silicon micro-needles, biodegradable, hydrophilic substances, substances that soften and/or change once in contact with the body and/or bodily fluids, or the like. In other embodiments, the dual insertion set may include multiple piercing members, only one piercing member, and/or no piercing members. In still further embodiments, the piercing member can replace the cannula and remain in the body to deliver fluids.

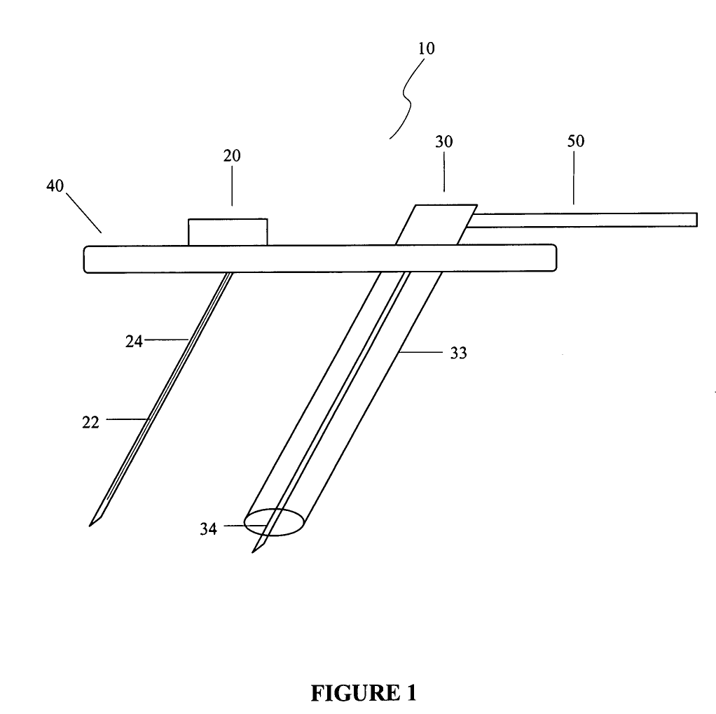

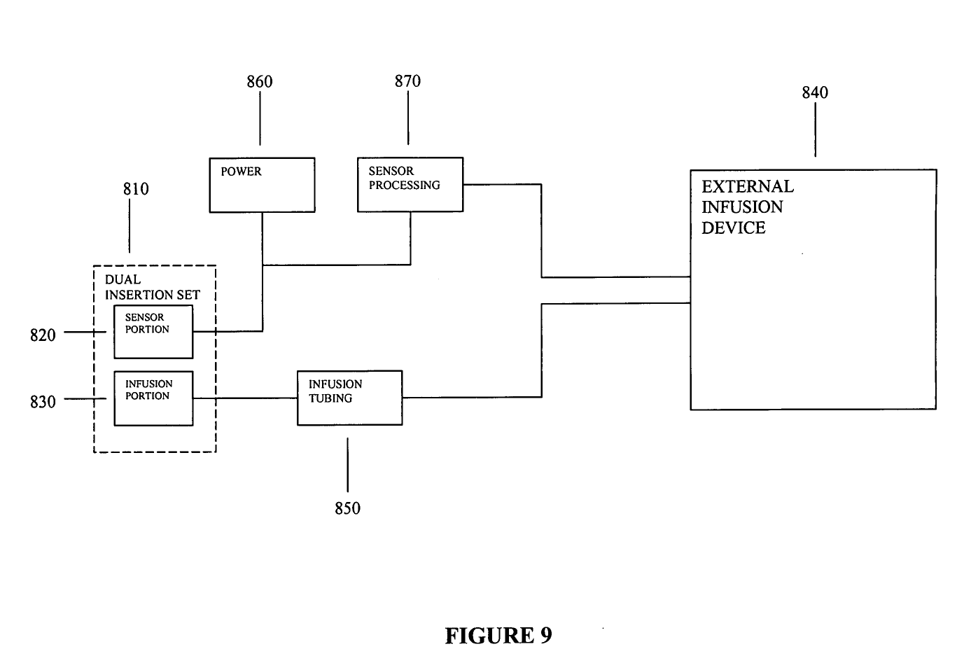

As illustrated in FIG. 1, a dual insertion set 10 in accordance with an embodiment of the present invention includes a sensor portion 20, an infusion portion 30, a base 40, a sensor 22, a cannula 33, and piercing members 24 and 34. Both portions 20 and 30 of the dual insertion set 10 are secured to base 40. Infusion portion 30 is connected at one end to tubing 50 that is connected to an external infusion device, pump or the like (FIG. 9). The sensor portion 20 is particularly designed for facilitating accurate placement of a sensor, i.e., a flexible thin film electrochemical sensor of the type used for monitoring specific blood parameters representative of a patient condition, as described in U.S. Pat. No. 5,391,250 entitled “Method of Fabricating Thin Film Sensors” and U.S. Pat. No. 6,484,046 entitled “Electrochemical Analyte Sensor,” both of which are specifically incorporated by reference herein. In some embodiments, the sensor portion 20 is used to monitor blood glucose levels in diabetic patients as described in U.S. Pat. Nos. 5,390,671, 5,568,806 and 5,586,553, entitled “Transcutaneous Sensor Insertion Set,” all of which are specifically incorporated by reference herein.

In the illustrated embodiment, sensor portion 20 of the dual insertion set 10 is provided for placement of a sensor 22 at a selected sensor placement site within the body of a patient. Sensor portion 20 includes a rigid hollow sensor insertion needle 24 for placement of the sensor distal segment having one or more exposed sensor electrodes. Insertion needle 24 is withdrawable to leave the sensor 22 electrodes in place at the selected site. In some embodiments, the sensor may be made from a substrate with notches cut in the substrate to from a necked down region in the substrate, as described in U.S. Pat. No. 6,484,045 entitled “Analyte Sensor and Method of Making the Same” and U.S. Patent Application Publication No. 20020032374 entitled “Improved Analyte Sensor and Method of Making the Same,” both of which are specifically incorporated by reference herein.

The sensor 22 is carried by base 40 adapted for placement on the patient's skin. Base 40 includes an enlarged and generally rectangular pad having an underside surface coated with a suitable pressure sensitive adhesive. The base may come in other shapes including, but not limited to, circular, square, triangular, trapezoidal, octagonal, or the like. The base may also come in various sizes. A peel off paper may be provided to cover and protect the adhesive layer until the dual insertion set 10 is ready for use. In alternative embodiments, the base 40 may be affixed to a suitable adhesive material that can hold the dual insertion set to the body.

The dual insertion set 10 may be designed to place the sensor 22 and cannula 33 subcutaneously or at another selected site within the body of a patient, in a manner minimizing patient discomfort and trauma. In the illustrated embodiment, the sensor 22 and cannula 33 are inserted into the body of the patient using piercing members 24 and 34. However, additional embodiments may include only one piercing member used to insert sensor 22 and cannula 33 into the body of the patient. In still further embodiments multiple needles, micro-needles or the like may be used to insert sensor 22 and cannula 33.

The infusion portion 30 of the illustrated embodiment may be used for delivering fluid, liquid, medication or the like to a patient on a continuous and/or programmable basis over an extended period of time, such as, for example, the administration of insulin to a diabetic patient by means of programmable external infusion device (FIG. 9). In some embodiments, the infusion portion 30 of the dual insertion 10 is of the type described in U.S. Pat. No. 4,755,173 entitled “Soft Cannula Subcutaneous Injection Set,” which is specifically incorporated by reference herein. Other embodiments may be formed similar to those described in U.S. Pat. No. 6,017,328 entitled “Device For Subcutaneous Medication Delivery,” U.S. Pat. No. 5,968,011 entitled “Subcutaneous Injection Set,” U.S. Pat. No. 6,086,575 entitled “Subcutaneous Infusion Device,” and U.S. Pat. No. 6,736,797 entitled “Subcutaneous Infusion Set,” all of which are specifically incorporated by reference herein. Alternative embodiments may include a cannula with the added capability to withdraw fluids from the body of the patient.

In use, the dual insertion set 10 of the illustrated embodiment permits accurate placement of the sensor 22 and cannula 33 at selected sensor and infusion placement sites within the body of the patient in a manner minimizing patient discomfort and trauma. More specifically, a peel-off paper may be removed from the pad at which time the base 40 can be pressed onto and seated upon the patient skin. During this step, insertion needles 24 and 34 pierce the patient's skin and carry the sensor 22 along with the cannula 33 to the appropriate sensor and infusion placement sites in the body of the patient. After the dual insertion set 10 is placed onto the skin of the patient, needles 24 and 34 can be withdrawn from the patient. During this withdrawal step, sensor insertion needle 24 slides over sensor 22 leaving the sensor 22 in direct contact with the patient's bodily fluid. The infusion needle 34 may be withdrawn from within the cannula 33, leaving the cannula 33 in a sub-dermal location within the body of the patient. Alternative embodiments place the sensor and/or cannula in the transcutaneous, subcutaneous, dermal, inter-peritoneal or peritoneal tissue of the patient. In still further embodiments, the sensor and/or cannula may be placed in the body of the patient using insertion tools of the type described in U.S. Pat. No. 6,093,172 entitled “Injector For A Subcutaneous Insertion Set,” U.S. Pat. No. 6,293,925 entitled “Insertion Device for an Insertion Set and Method of Using the Same,” and U.S. Pat. No. 6,607,509 entitled “Insertion Device for an Insertion Set and Method of Using the Same,” all of which are specifically incorporated by reference herein.

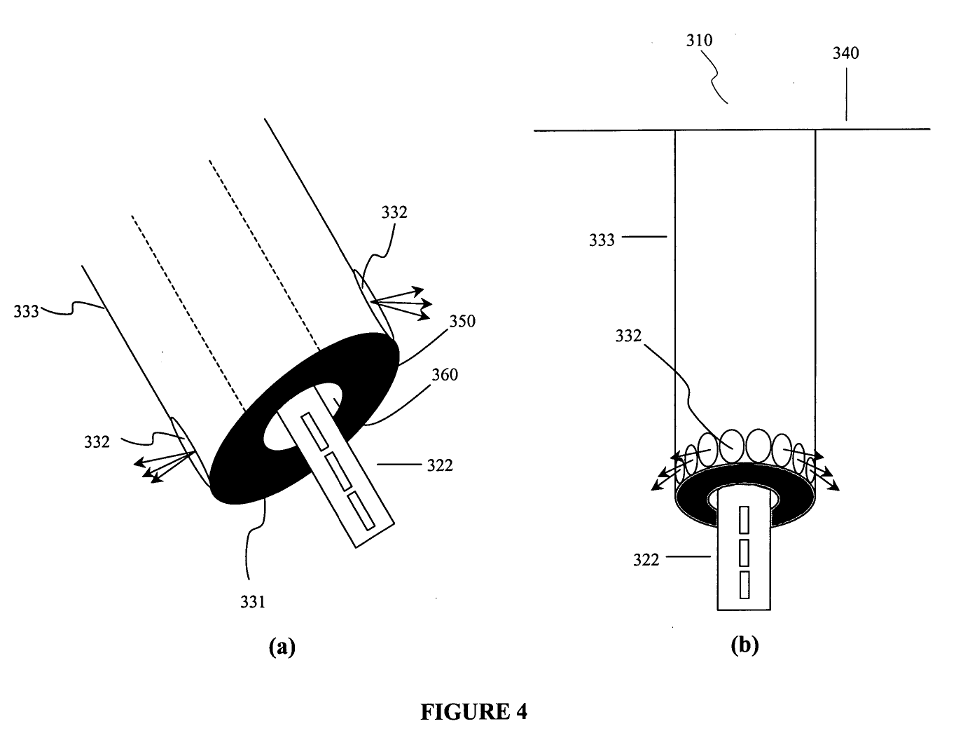

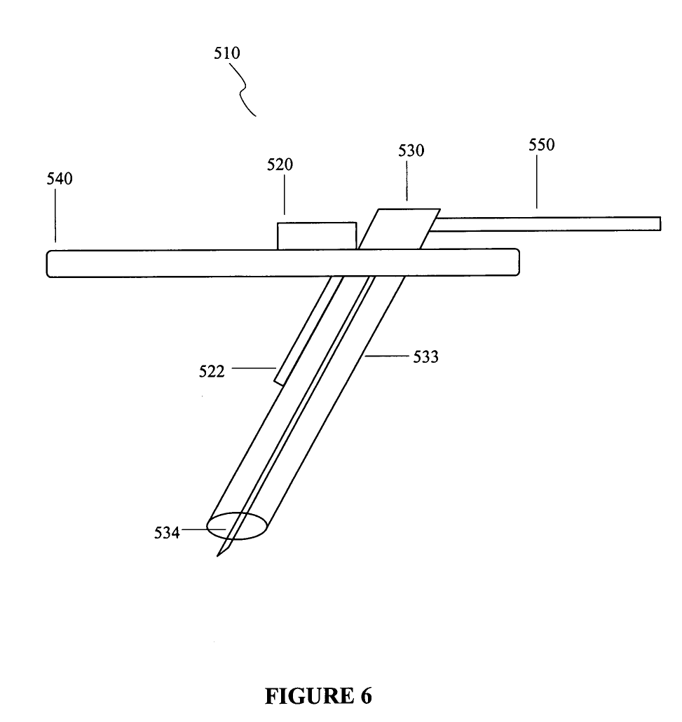

The sensor 22 and cannula 33 of the illustrated embodiment may be laterally spaced apart from each another. In some embodiments, the lateral distance between the sensor 22 and cannula 33 may be in the range of 10 mm-40 mm, or the like, or other ranges there between. Alternative embodiments may use smaller displacements from 0-10 mm. In still other embodiments, the sensor 22 and cannula 33 may be laterally spaced side-by-side (FIG. 4, FIG. 6). Alternate embodiments may space the sensor 22 and cannula 33 axially, diagonally, or the like. In still other embodiments, the length of cannula 33 may be in the range of 3 mm-12 mm, or the like, or other ranges there between. In further embodiments, the length of cannula 33 may be less than 3 mm and/or greater than 12 mm. In other embodiments, the insertion angle of cannula 33 may vary depending on certain factors including but not limited to body type, body weight, placement site location and the like. These angles range from 0 degrees to 90 degrees relative to the base. In still further embodiments, the length and position of sensor 22 may vary in a similar fashion to the variations of cannula 33 in previous embodiments.

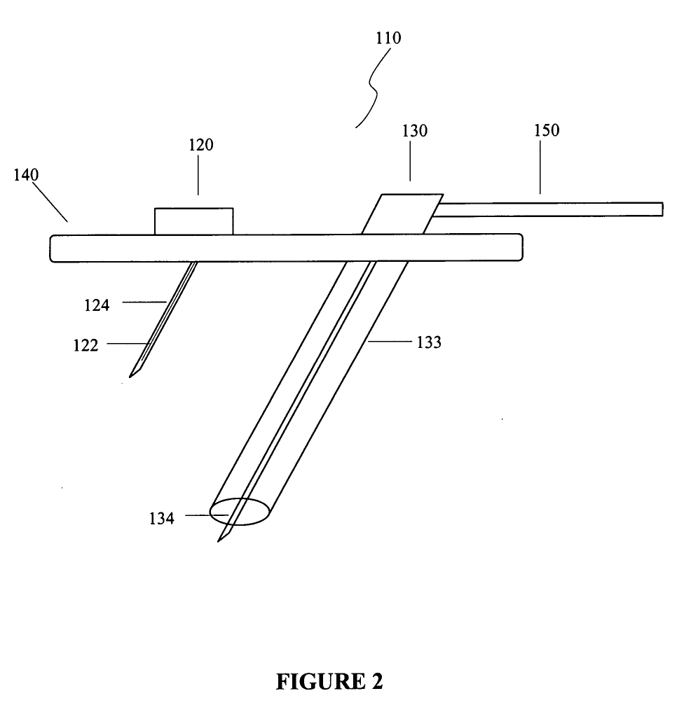

As shown in FIG. 2, a dual insertion set 110 in accordance with an embodiment of the present invention includes a sensor portion 120, an infusion portion 130, a base 140, a sensor 122, a cannula 133, and piercing members 124 and 134. Both portions 120 and 130 of the dual insertion set 110 are secured to base 140, similar to the previous embodiments. However, in the illustrated embodiment, the length of sensor portion 120 is smaller, compared to the length of the infusion portion 130. In this embodiment, the sensor 122 may be placed at a different depth in the body of the patient, due its shorter size. In other embodiments, the cannula 133 maybe sized shorter in length relative to the sensor 122. The variations in length of the sensor 122 and the cannula 133 are determined by many factors including, but not limited to, the sensor and infusion placement site location on the body of the patient, the patient's relative sensitivity in the sensor and infusion placement site location, and the like. Additionally, in the illustrated embodiment, sensor piercing member 124 and infusion piercing member 134 may be sized according to the length of sensor 122 and cannula 133. In some embodiments, the piercing members 124 and 134 are sized slightly longer than the sensor 122 and cannula 133.

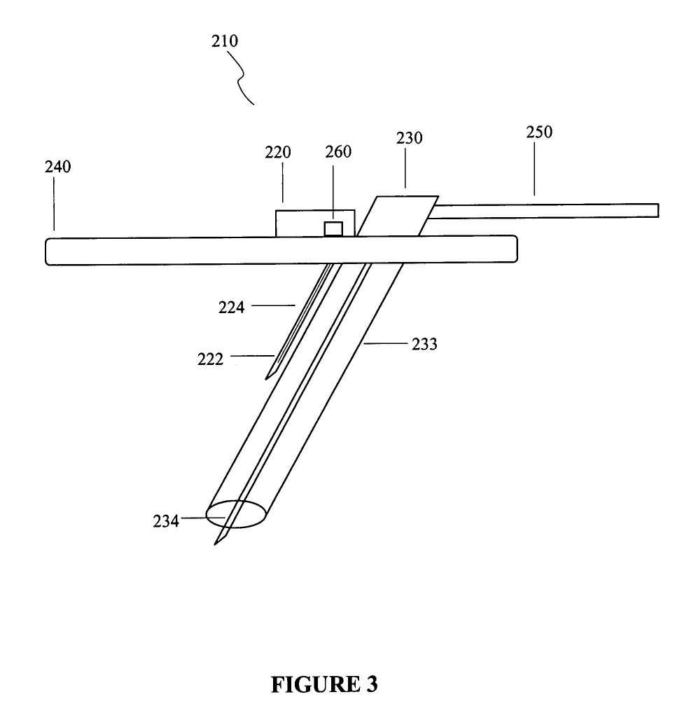

As shown in FIG. 3, a dual insertion set 210 in accordance with an embodiment of the present invention includes a sensor portion 220, an infusion portion 230, a base 240, a sensor 222, a cannula 233, and piercing members 224 and 234. The sensor portion 220 may also include a power supply 260, positioned on the base 240 above the sensor 222. The inclusion of a power supply may allow the sensor to transmit readings to an external infusion device (see FIG. 9). In alternative embodiments, the power supply may allow the sensor to transmit readings to a glucose monitor, a medication device, a PDA, a PC, a laptop, the internet, or the like. In additional embodiments, the power supply 260 may provide the capability to use the power for a leak detection system at the placement site as described in U.S. Pat. No. 6,461,329 entitled “Infusion Site Leak Detection System and Method of Using the Same,” which is specifically incorporated by reference herein. In other embodiments of the invention, the power supply 260 may be used to power an alarm or similar feature added to the dual insertion set. In additional embodiments, the power supply may be removable to allow use of the power supply on different insertion sets. In these embodiments, the power supply may attach or clip onto the dual insertion using various methods known in the art.

As shown in FIGS. 4(a) and 4(b), a dual insertion set 310 in accordance with another embodiment of the present invention includes a base 340, a sensor 322, a cannula 333 with openings 332. The cannula 333 further includes an outer lumen 350 and an inner lumen 360. The outer lumen of cannula 333 may be sealed at its distal end 331. In the illustrated embodiment, the dual insertion set 310 may use a single piercing member (not shown) to place the cannula 333 and the sensor 322 at a single placement site in the body of the patient. The outer lumen 350 is for infusing the fluid into the body of the patient through the openings 332. The inner lumen 360 may contain the sensor 322. Since the outer lumen 350 is sealed at its distal end 331 infused fluid avoids contact with the sensor 322. The sensor 322 protrudes out of the inner lumen 350 to monitor a body characteristic. In use, fluid enters the outer lumen 350 of cannula 333 and is dispensed into the body of the patient through openings 332 located around cannula 333. In this configuration, sensor 322 is not affected by infused insulin because the outer lumen 350 is sealed off at the distal tip 331 and the fluid is dispersed into the body of the patient via openings 332 away from the sensor 322. In alternative embodiments, the openings 332 may be replaced and/or used in combination with more or less openings, windows, porous membranes or the like.

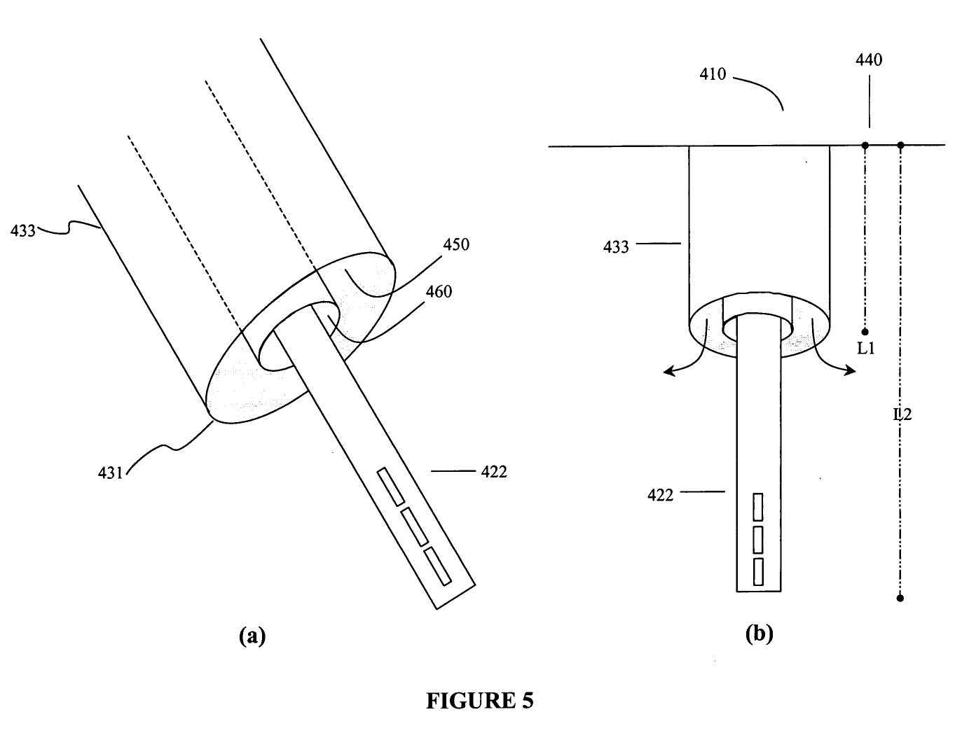

As shown in FIGS. 5(a) and 5(b), a dual insertion 410 in accordance with yet another embodiment of the present invention includes a base 440, a sensor 422 and a cannula 433. The cannula 433 further includes an outer lumen 450 and an inner lumen 460. The outer lumen of cannula 433 may be open at its distal end 431. In the illustrated embodiment, the dual insertion set 410 uses a single piercing member (not shown) to place the cannula 433 and the sensor 422 at a single placement site in the body of the patient. The outer lumen 450 is for infusing fluid into the body of the patient through its distal end opening 431. The inner lumen 460 contains the sensor 422. The sensor 422 protrudes out of the inner lumen 450 to monitor a body characteristic. The sensor 422 is positioned a predetermined distance L2 into the body of the patient. In the illustrated embodiment, the cannula 433 is much shorter in length, having a length L1. In use, the sensor 422 is placed deep within the sub-dermal portion of the body to achieve accurate body characteristic readings, while insuring no interference from the infused fluid from cannula 433. Having distal end 431 open allows the infused fluid to enter the body of the patient directly through the outer lumen of the cannula 433. This embodiment may be easier to manufacture than the previously described embodiment in FIGS. 4(a) and 4(b). The placement depth characteristics L1 and L2 range from 3 mm to 12 mm in size, depending on the type of fluid/sensor combination being used. In other embodiments, shorter and/or longer dimensions may be utilized including less than 3 mm and/or greater than 12 mm.

As shown in FIG. 6, a dual insertion set 510 in accordance with another embodiment of the present invention includes a sensor portion 520, an infusion portion 530, a base 540, a sensor 522, a cannula 533, and single piercing member 534. In the illustrated embodiment, the sensor 522 directly attaches to the cannula 533. In some embodiments, the sensor 522 may lay along side an outer wall of the cannula 533, while other embodiments may have a flexible sensor concentrically surrounding the diameter of the cannula 533. The single piercing member 534 inserts the sensor 522 and the cannula 533 into the body of the patient, eliminating the need for multiple piercing members, thus minimizing patient discomfort and trauma. In alternative embodiments, the sensor may be made from a substrate with notches cut in the substrate to from a necked down region in the substrate, as described in U.S. Pat. No. 6,484,045 entitled “Analyte Sensor and Method of Making the Same” and U.S. Patent Application Publication No. 20020032374 entitled “Improved Analyte Sensor and Method of Making the Same,” both of which are specifically incorporated by reference herein.

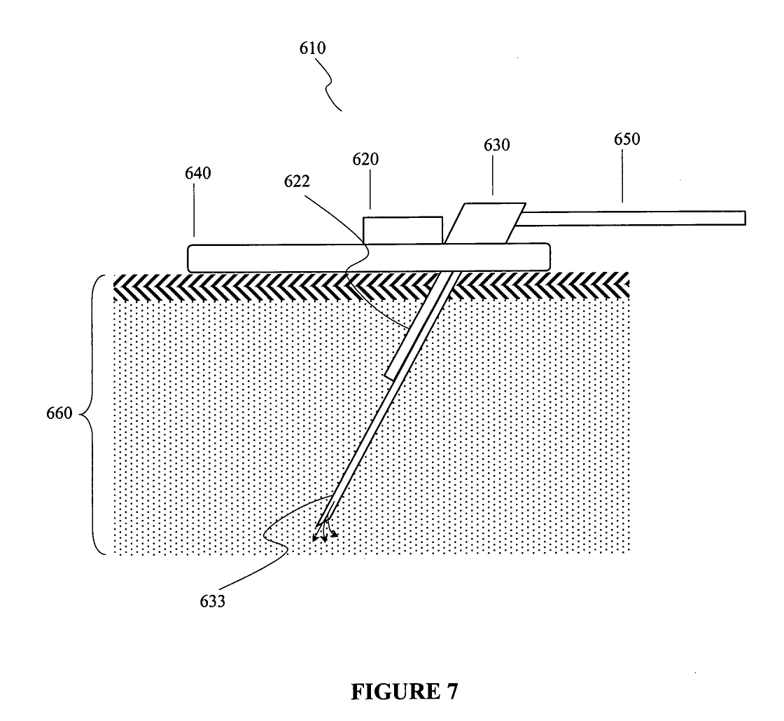

As shown in FIG. 7, a dual insertion set 610 in accordance with yet another embodiment of the present invention includes a sensor portion 620, an infusion portion 630, a base 640, a sensor 622 and a piercing member 633. The cannula 133 (FIG. 1) may be removed entirely from the dual insertion set 610, being replaced by a piercing member 633. The piercing member 633 may serve the dual function of inserting the sensor into the body of the patient along with delivering fluid into the body of the patient. In the illustrated embodiment, the piercing member 633 may be a hollow, thin needle having a diameter in the range of 18 gauge to 33 gauge. The needle 633 may be a metal needle, hollow, solid, half needle (or other fraction) or the like. In use, the piercing member 633 can puncture the skin of the patient and deliver fluids to the patient, similar to the cannula of the previous embodiments. Furthermore, piercing member 633 may be capable of remaining in the body of the patient for an extended period of time, without causing noticeable discomfort. In alternative embodiments, the sensor may be applied and/or formed directly onto the piercing member as described in previous embodiments.

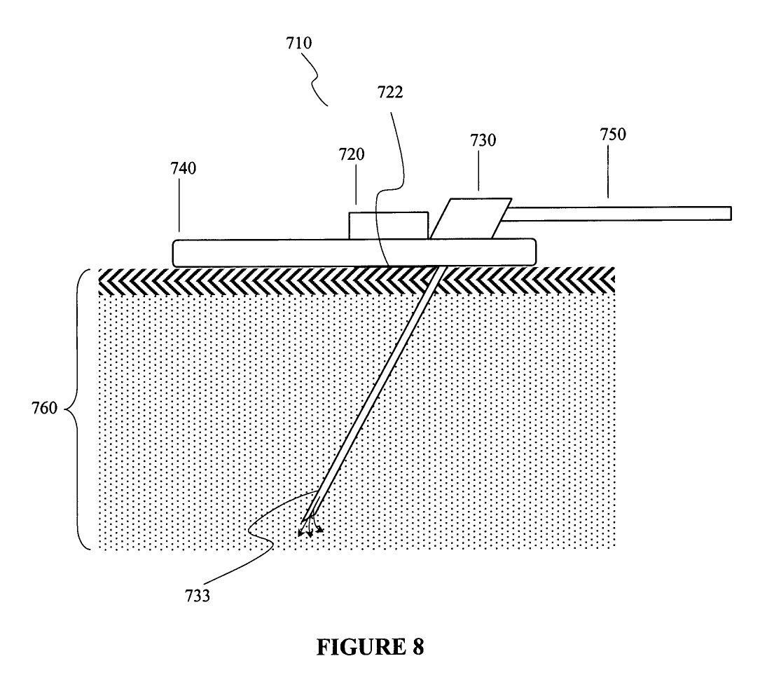

As shown in FIG. 8, a dual insertion set 710 in accordance with another embodiment of the present invention includes a sensor portion 720, an infusion portion 730, a base 740, a sensor 722 and a piercing member 733. The sensor 722 is placed underneath the base 740. The cannula 133 (FIG. 1) may be removed entirely from the dual insertion set 710, being replaced by a piercing member 733. In the illustrated embodiment, the piercing member 733 can deliver fluids as well as withdraw fluids from the body of the patient. Fluid is withdrawn from the patient via piercing member 733 and passes through sensor 733 where it is analyzed to determine a body characteristic level, i.e., blood glucose levels in a diabetic patient. In the illustrated embodiment, the piercing member may be replaced and/or used in conjunction with a cannula as described in previous embodiments.

In still a further embodiment shown in FIG. 9, a dual insertion set 810 is shown in a block diagram configuration connected to a system that includes a power supply 860, a sensor processing module 870, an external infusion device 840 and infusion tubing 850. The dual insertion set 810 includes a sensor portion 820 and an infusion portion 830. The infusion portion 830 is coupled to infusion tubing 850 that receives fluid from the external infusion device 840. The sensor portion 820 is coupled to the power supply 860 and the sensor processing module 870, which are both coupled to the external infusion device 840.

While the description above refers to particular embodiments of the present invention, it will be understood that many modifications may be made without departing from the spirit thereof. The accompanying claims are intended to cover such modifications as would fall within the true scope and spirit of the present invention.

The presently disclosed embodiments are therefore to be considered in all respects as illustrative and not restrictive, the scope of the invention being indicated by the appended claims, rather than the foregoing description, and all changes which come within the meaning and range of equivalency of the claims are therefore intended to be embraced therein.

What is claimed is:

1. A dual insertion set for supplying a fluid to a body of a patient and for monitoring a body characteristic of the patient, the dual insertion set comprising:
a base adapted to secure the dual insertion set to the skin of a patient;
an infusion portion including at least one cannula for supplying a fluid to an infusion placement site, wherein the at least one cannula is coupled to and extends from the base, wherein the at least one cannula has at least one lumen with a distal end for fluid communication with the infusion placement site, and wherein the at least one cannula has at least one port structure formed near another end of the at least one lumen opposite the distal end; and
a sensor portion including at least one sensor coupled to and extending from the base and having at least one sensor electrode formed on a substrate, wherein the at least one sensor is for determining at least one body characteristic of the patient at a sensor placement site.

2. A dual insertion set according to claim 1, including at least one piercing member coupled to and extending from the base to facilitate insertion of at least one of the at least one cannula and the at least one sensor.

3. A dual insertion set according to claim 2, wherein two piercing members are used to insert the at least one cannula and the at least one sensor into the body of the patient.

4. A dual insertion set according to claim 1, wherein the infusion portion and the sensor portion are spaced a predetermined distance apart from one another.

5. A dual insertion set according to claim 4, wherein the two piercing members are metal needles.

6. A dual insertion set according to claim 1, wherein the length of the sensor portion is sized shorter relative to the length of the infusion portion.

7. A dual insertion set according to claim 1, wherein the length of the infusion portion is sized shorter relative to the length of the sensor portion.

8. A dual insertion set according to claim 1, wherein the cannula and the sensor are equal in length.

9. A dual insertion set according to claim 1, wherein one piercing member is used to insert the dual insertion set into the body of the patient.

10. A dual insertion set according to claim 9, wherein the piercing member is a metal needle.

11. A dual insertion set according to claim 1, wherein the at least one cannula includes an outer lumen to supply the fluid and an inner lumen to contain the sensor portion.

12. A dual insertion set according to claim 11, wherein the outer lumen of the at least one cannula is sealed off at the distal end, wherein the inner lumen of the at least one cannula is open to allow the at least one sensor to protrude out of the inner lumen, and wherein the at least one cannula includes at least one opening for infusing the fluid into the body of the patient.

13. A dual insertion set according to claim 1, wherein the at least one sensor includes at least one internal power supply.

14. A dual insertion set according to claim 1, wherein the at least one sensor includes at least one removable power supply.

15. A dual insertion set according to claim 1, wherein the at least one sensor includes a leak detection system.

16. A dual insertion set according to claim 1, wherein the infused fluid is insulin.

17. A dual insertion set according to claim 1, wherein the at least one monitored body characteristic is blood glucose.

18. A dual insertion set for supplying a fluid to a body of a patient and for monitoring a body characteristic of the patient, the dual insertion set comprising:
a base adapted to secure the dual insertion set to the skin of a patient;
an infusion portion including at least one cannula for supplying a fluid to an infusion placement site, wherein the at least one cannula is coupled to and extends from the base, wherein the at least one cannula has at least one lumen with a distal end for fluid communication with the infusion placement site, and wherein the at least one cannula has at least one port structure formed near another end of the at least one lumen opposite the distal end;
a sensor portion including at least one sensor having at least one sensor electrode formed on a substrate, wherein the at least one sensor is for determining at least one body characteristic of the patient at a sensor placement site,
wherein the at least one cannula includes an outer lumen to supply the fluid and an inner lumen to contain the sensor portion.

19. A dual insertion set according to claim 18, including a piercing member coupled to and extending from the base to facilitate insertion of at least one of the at least one cannula and the at least one sensor.

20. A dual insertion set according to claim 19, wherein the piercing member is a metal needle.

21. A dual insertion set according to claim 18, wherein the outer lumen of the at least one cannula is sealed at the distal end, and wherein the inner lumen of the at least one cannula is open to allow the at least one sensor to protrude out of the inner lumen.

22. A dual insertion set according to claim 18, wherein the outer lumen of the at least one cannula includes at least one opening for infusing the fluid into the body of the patient.

23. A dual insertion set according to claim 18, wherein the infused fluid is insulin.

24. A dual insertion set according to claim 18, wherein the at least one monitored body characteristic is blood glucose.

25. A dual insertion set according to claim 18, wherein the at least one sensor includes an internal power supply.

26. A dual insertion set according to claim 18, wherein the at least one sensor includes at least one removable power supply.

27. A dual insertion set according to claim 18, wherein the at least one sensor includes a leak detection system.

28. A dual insertion set for supplying a fluid to a body of a patient and for monitoring a body characteristic of the patient, the dual insertion set comprising:
a base adapted to secure the dual insertion set to the skin of a patient;
an infusion portion including at least one cannula for supplying a fluid to an infusion placement site, wherein the at least one cannula is coupled to and extends from the base, wherein the at least one cannula has at least one lumen with a distal end for fluid communication with the placement site, and wherein the at least one cannula has at least one port structure formed near another end of the at least one lumen opposite the distal end;
a sensor portion including at least one sensor having at least one sensor electrode formed on a substrate, wherein the at least one sensor is for determining at least one body characteristic of the patient at a sensor placement site,
wherein the at least one sensor is coupled to an outer wall of the at least one cannula.

29. A dual insertion set according to claim 28, including a piercing member coupled to and extending from the base to facilitate insertion of at least one of the at least one cannula and the at least one sensor.

30. A dual insertion set according to claim 29, wherein the piercing member is a metal needle.

31. A dual insertion set according to claim 28, wherein the infused fluid is insulin.

32. A dual insertion set according to claim 28, wherein the at least one monitored body characteristic is blood glucose.

33. A dual insertion set according to claim 28, wherein the at least one sensor includes an internal power supply.

34. A dual insertion set according to claim 28, wherein the at least one sensor includes at least one removable power supply.

35. A dual insertion set according to claim 28, wherein the at least one sensor includes a leak detection system.

36. A dual insertion set for supplying a fluid to a body of a patient and for monitoring a body characteristic of the patient, the dual insertion set comprising:
a base adapted to secure the dual insertion set to the skin of a patient;
an infusion portion including a piercing member for penetrating the skin of the patient and for supplying a fluid to an infusion placement site, wherein the piercing member is coupled to and extends from the base, wherein the piercing member has at least one lumen with a distal end for fluid communication with the placement site, and wherein the piercing member has at least one port structure formed near another end of the at least one lumen opposite the distal end; and
a sensor portion including at least one sensor coupled to and extending from the base having at least one sensor electrode formed on a substrate, wherein the at least one sensor for to determining at least one body characteristic of the patient at the placement site.

37. A dual insertion set according to claim 36, wherein the at least one sensor is coupled to an outer wall of the piercing member.

38. A dual insertion set for supplying a fluid to a body of a patient and for monitoring a body characteristic of the patient, the dual insertion set comprising:
a base adapted to secure the dual insertion set to the skin of a patient;
an infusion portion including a piercing member for penetrating the skin of the patient and for supplying a fluid to an infusion placement site, wherein the piercing member is coupled to and extends from the base, wherein the piercing member has at least one lumen with a distal end for fluid communication with the placement site, and wherein the piercing member has at least one port structure formed near another end of the at least one lumen opposite the distal end;
a sensor portion including at least one sensor having at least one sensor electrode formed on a substrate, wherein the at least one sensor for determining at least one body characteristic of the patient at a sensor placement site;
wherein the at least one sensor is placed on the skin of the patient; and
wherein the piercing member withdraws fluid from the body of the patient to provide fluid contact with the at least one sensor electrode.
